(19) United States
(12) Patent Application Publication
(10) Pub. No.: US 2014/0235480 A1
(43) Pub. Date: Aug. 21, 2014

(54) Ultra-sensitive Detection of Analytes

(71) Applicant: Craig David Shimasaki, Edmond, OK (US)

(72) Inventor: Craig David Shimasaki, Edmond, OK (US)

(21) Appl. No.: 14/176,440

(22) Filed: Feb. 10, 2014

Related U.S. Application Data
(60) Provisional application No. 61/763,050, filed on Feb. 11, 2013.

(51) Int. Cl. G01N 33/543

(52) U.S. Cl. CPC G01N 33/54373
USPC 506/9; 422/69; 436/501; 506/18; 435/5; 435/287.2

(57) ABSTRACT

The present invention provides devices and methods for ultra-sensitive detection of analytes of interest in a rapid and convenient manner. A portable handheld device having replaceable cartridges adaptable for detection of a wide array of analytes (e.g., biological, environmental, and the like) is provided. Sample wells containing bound capture antibodies are provided with microelectrical circuits across each of the sample wells such that there is a nanoscale gap which prevents passage of electrical current. A sample potentially containing the analyte of interest is pumped into a sample well containing bound capture antibodies directed to an first epitope of the analyte of interest. After removal of unbound sample, a metal-labeled antibody directed to a second epitope of the analyte of interest thereby, if the analyte of interest was present in the sample, a metal-labeled antibody-analyte-capture antibody complex remains bound to the sample well. Passage of electrical current across the nanoscale gap (as indicated by a change in electrical resistance, capacitance, impedance and/or conductance, as well as combinations thereof, as compared to the blank control well) thereby reveals the presence of the metal-labeled antibody-analyte-capture antibody complex and, thus, the presence of the analyte of interest in the sample.

### RELATED APPLICATION

This application is based on, and claims benefit of, U.S. Provisional Application 61/763,050, filed on Feb. 11, 2013, which is hereby incorporated by reference.

### FIELD OF THE INVENTION

The present invention provides devices and methods for ultra-sensitive detection of analytes of interest in a rapid and convenient manner. A portable handheld device having replaceable cartridges adaptable for detection of a wide array of analytes (e.g., biological, environmental, and the like) is provided.

### BACKGROUND OF THE INVENTION

Detection of analytes and diagnostic methods require that a specific analyte of interest be identified within a sample of interest (e.g., human or other biological specimen or environmental sample). The utility of such methods are limited by their sensitivity and/or ability to detect an analyte at a specific concentration or quantity contained within the sample. If the concentration of the analyte in the sample is below the limit of detection for the device or method, the sample is generally reported as “below the limits of detection.” Determination of levels below the detection limits generally require the use of laboratories having more sophisticated and expensive instrumentation with increased sensitivity. Results obtained from such instrumentation often require significant time delays. Thus, if real-time results are desired or required, such options with their time delays are not generally acceptable.

Methods of detection for analytes have been in existence for decades. Analytes can be whole or parts of organisms, molecules (biological or non-biological) or chemical (toxic or hazardous) or environmental compounds or other entities. Often, there is a need to determine the specific analyte that is present in a particular sample. For instance, in the medical industry, various types of clinical specimens such as blood, urine, saliva or spinal fluid are taken from patients to determine the presence or absence of certain types of viruses, bacteria, enzymes, or biological or biochemical molecules present within the sample. Often, the concentration of these analytes is in such low quantities that certain diagnostic testing methods—especially those methods designed for field or point of use testing—do not have the sensitivity to accurately determine their presence or absence. Various improvements have been commercially made to increase the level of detection of various analytes. For example, DNA amplification or nucleic acid duplication methods such as PCR (polymerase chain reaction) repetitively duplicates copies of the original DNA molecule contained within a sample until a high enough concentration of DNA is reached and subsequently can be identified by existing methods of detection. These types of amplification and duplication methods operate on large, bulky and costly instruments which are purchased and operated by clinical laboratories specializing in testing for these analytes.

Detection of analytes and diagnostic methods for field or point of use testing require that a specific analyte of interest be identified within a sample of interest (e.g., human or other biological specimen or environmental sample). The utility of such methods are limited by their sensitivity and/or ability to detect an analyte at a specific concentration or quantity contained within the sample. If the concentration of the analyte in the sample is below the limit of detection for the device or method, the sample is generally reported as “below the limits of detection.” Determination of levels below the detection limits generally require the use of laboratories having more sophisticated and expensive instrumentation with increased sensitivity as discussed above. Results obtained from such instrumentation often require significant time delays. Thus, if real-time results are desired or required, such options with their time delays are not generally acceptable.

Devices (especially portable and handheld devices) and methods primarily designed for use in the field or in point of use environments (e.g., doctor's offices) are generally less sensitive than more sophisticated and expensive laboratory instrumentation. Thus, “below the limits of detection” samples evaluated using such field or point of use devices generally must be sent to a suitable testing facility, thereby incurring additional costs and, more importantly in many cases, delays in obtaining the desired results. In many cases, delays in obtaining results can result in delays in determining and then beginning viable treatment options. Indeed, such delays can be significant and can result in significant reduction in the effectiveness of treatment options.

There remains a need to have a highly sensitive, rapid, point-of-care testing platform for various analytes without having to send a specimen out to a commercial laboratory for a delayed diagnostic result. It is desired, therefore, to provide devices (especially portable and handheld devices) and methods for use in the field or in point of use environments (e.g., doctor's offices) having increased sensitivity. The present invention provides such devices and methods.

### SUMMARY OF THE INVENTION

The present invention provides for an ultra-sensitive method of detection that is convenient and avoids the need to send out specimens for detection that contain analytes too low for existing field use or point-of-care environments. The present invention provides for a rapid, convenient, and ultra-sensitive method of detection of analytes of interest, especially for biological and environmental analytes. The device and method of this invention are suitable for use in the field, laboratory, or point of care (e.g., doctor offices) environments. The sensitivity of the present device and method is superior to currently available devices or methods for use in the field or point-of-care environments and generally avoids delays in obtaining results associated with sending samples to a testing laboratory. The present invention provides for an ultra-sensitive method of detection that is convenient and, in most cases, avoids the need to rely on commercial laboratories for detection of analytes present at levels too low for existing field use or point-of-care devices or methods.

The present invention provides a device for detection of one or more analytes of interest in a biological or environmental sample, said device comprising
a device body and an interchangeable and replaceable platform chip or cartridge, wherein the device body is adapted to accept the interchangeable and replaceable platform chip;
wherein the device body comprises a battery, input means, and readout or output means;
wherein the platform chip or cartridge comprises
   (1) a sample reservoir, a reservoir for a metal-labeled antibody, and at least one wash solution reservoir, wherein the metal-labeled antibody is specific to a first epitope of the one or more analytes of interest and the metal-labeled antibody contains an electrically conductive metal bound thereto;
   (2) a plurality of sample wells containing an area of capture antibodies specific to a second epitope of the one or more analytes of interest, wherein the capture antibodies are bound to the sample wells;
   (3) individual microelectrical circuits across each of the sample wells such that there is a nanoscale gap for each of the plurality of sample wells in the area of the capture antibodies, wherein the nanoscale gap is about 10 to about 200 nanometers; and
   (4) at least one microfluidic pump adapted to individually move the sample, the metal-labeled antibody, and the one or more wash solution in a preselected order, through microfluidic channels into the sample wells at preselected times, hold within the sample wells for preselected periods, and remove from the sample wells at preselected times;
whereby the sample can be applied to the sample well to contact the capture antibodies and maintained therein for a sufficient time for analyte of interest, if present, to react with its corresponding capture antibody and be bound thereto to form an analyte-capture antibody complex, after which wash solution can be pumped through the sample well to remove unbound sample, after which metal-labeled antibody can be can be pumped into the sample well to contact any and maintained therein for a sufficient time for metal-labeled antibody to react with the analyte-capture antibody complex, if present, and be bound thereto form a metal-labeled antibody-analyte-capture antibody complex, after which wash solution can pumped through the sample well to remove unbound metal-labeled antibody, after which an electrical current can be passed across sample well such that if the analyte of interest was present, the metal-labeled antibody-analyte-capture antibody complex will allow the electrical current to pass over the nanoscale gap, thereby indicating the presence of the analyte of interest in the sample.

The present invention also provides a method for detecting of an analyte of interest in a biological or environmental sample, said method comprising

(1) applying the sample to a sample well containing bound capture antibodies, wherein the sample well has a microelectrical circuit attached to the sample well such that there is a nanoscale gap of about 10 to about 200 nanometers across the sample well in area of the capture antibodies, are wherein the bound capture antibodies are specific to a second epitope of the analyte of interest;

(2) maintaining the sample in contact with the bound capture antibody for a sufficient time for the analyte of interest, if present, to react with the capture antibody and be bound thereto to form an analyte-capture antibody complex;

(3) washing the sample well from step (2) to remove the unbound sample and leaving the analyte-capture antibody complex bound to the sample well;

(4) adding metal-labeled antibodies to the sample well in step (3), wherein the metal-labeled antibodies are specific to a first epitope of the analyte of interest and metal-labeled antibodies contain an electrically conductive metal bound thereto;

(5) maintaining the metal-labeled antibodies in contact with the analyte-capture antibody complex for a sufficient time for the analyte of interest, if present, to react with the analyte-capture antibody complex and be bound thereto to form a metal antibody-analyte-capture antibody complex;

(6) washing the sample well from step (5) to remove the unbound metal-labeled antibody and leaving the methal antibody-analyte-capture antibody complex bound to the sample well; and

(7) passing an electrical current across sample well and its microelectrical circuit such that if the analyte of interest was present, the metal-labeled antibody-analyte-capture antibody complex will allow the electrical current to pass over the nanoscale gap, thereby indicating the presence of the analyte of interest in the sample.

### BRIEF DESCRIPTION OF THE DRAWINGS

FIG. 1 provides a schematic view of the device of the present invention.

FIG. 2 provides a flowchart illustrating the method of the present invention.

FIG. 3 provides a schematic illustration of the detection method of the present invention.

FIG. 4 provides a second schematic illustration of the detection method of the present invention. FIG. 4A shows the nanoscale gap embedded within a single sample well containing bound capture antibodies Y. FIG. 4B shows the sample well after exposure to the sample containing the analyte and removal of excess sample by a first wash. Remaining in the well are bound analyte. FIG. 4C shows the sample well after exposure to the metal-labeled antibody and removal of excess metal-labeled antibody. Bound metal-labeled antibody retained by the bound analyte remain in the sample well.

FIG. 5 provides an illustration of one embodiment of the device of this invention.

### DETAILED DESCRIPTION OF THE INVENTION

The present invention provides for an ultra-sensitive method of detection that is convenient and avoids the need to send out specimens for detection that contain analytes too low for existing field use or point-of-care environments. The present devices comprises a hand-held portable detection device with interchangeable and replaceable nano-circuit microfluidic antibody detection platforms for insertion therein. The hand-held portable detection device measures electrical impulses and changes in capacitance, conductance or resistance. The interchangeable and disposable nano-circuit microfluidic antibody detection platform contains a specified panel of antibodies utilized in the detection of the antigen or analyte of interest. The platform cartridges are inserted into the detection device reader which contains actuators to pump fluids through microfluidic channels into detection wells containing the appropriate antibodies at the appropriate time intervals. The platform cartridges contain microfluidic channels that allow for various wash solutions and/or reagents that are to be applied to specific antibodies within the detection wells of the replaceable platform. The hand-held portable detection device (which receives the interchangeable and disposable detection platform) preferably contains electronics to recognize and control the detection platform based on input and instructions provided by the user. Preferably, the platform cartridges contains identifying information concerning the capture antibodies within each cells whereby the device can determine the appropriate parameters by which the analysis for each cell will be carried out. Such parameters include, for example, the temperatures, incubation times, and wash times for the analysis. Preferable, the device allows the transmission of data from the analysis to a storage device to allow the data and results obtained to be assessed or transferred as needed.

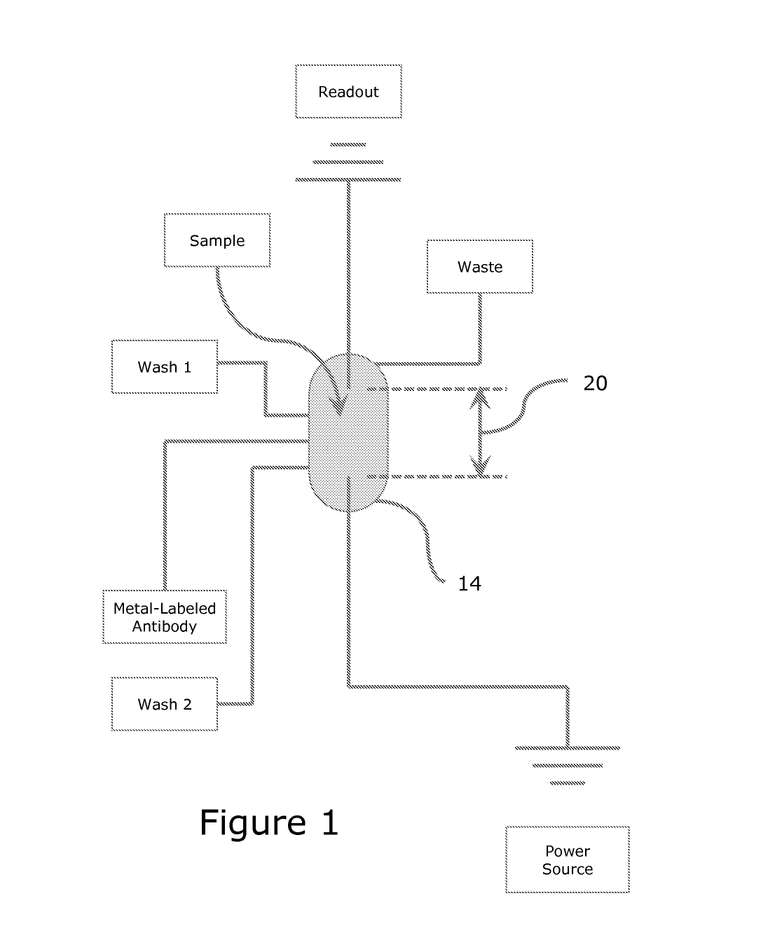

FIG. 1 provides a schematic view of the device of the present invention containing a single sample well 10. Although not specifically shown, sample well 10 contains a capture antibody (preferably a monoclonal capture antibody) specific for an epitope of the analyte of interest. An microelectrical circuit 14 is placed across the sample well 10 such that there exists a nanoscale gap (as indicated by dimension 2). The capture antibody is bound to or embedded within the sample well across the general area of this nanoscale gap 2. Using microfluidic pumping techniques, a sample potentially containing the analyte of interest can be injected into the sample well in order to contact the capture antibody bound therein. If present, the analyte of interest, after a suitable incubation period, will be bound to the capture antibody. After such an incubation period, the sample along with any unbound analyte of interest is removed from the sample well using wash solution 1 to flush excess sample to waste (again using microfluidic pumping techniques). Assuming the analyte of interest was present in the sample, a portion of the analyte will be bound the capture antibody and remain within the sample well 10.

A metal-labeled antibody (preferably a metal-labeled monoclonal antibody) is then injected into the sample well 10 using microfluidic pumping techniques. This metal-labeled antibody is also specific to an epitope of the analyte of interest. The epitopes to which the metal-labeled antibody and the capture antibody are specific may be the same or different epitopes on the analyte of interest. Preferably, the metal-labeled antibody and the capture antibody are specific to different epitopes on the analyte of interest. After a suitable incubation time, the metal-labeled antibody will be bound to a portion of the analyte which is in turn bound to the capture antibody which is in turn attached to the sample well. After such an incubation period, unbound metal-labeled antibody is removed from the sample well using wash solution 2 to waste (again using microfluidic pumping techniques). Again assuming the analyte of interest was present in the sample, a “sandwich” comprising metal-labeled antibody, the analyte of interest, and the capture antibody will remain the sample well. An electrical current is then applied through circuit 14 and, if the metal-labeled antibody is present, across the nanoscale gap 20 and can be detected by the device. In effect, the presence of metal bound to the metal-labeled antibody allows the current to pass across the nanoscale gap 20 and to detect the presence of the analyte of interest in the sample. Should the analyte of interest not be present in the sample, the metal-labeled antibody should not be retained in the sample well 20 and thus current should not pass across the nanoscale gap 20. Detection of the current passing across the nanoscale gap thus indicates the presence of the analyte of interest in the sample.

Although not shown in FIG. 1, it is generally preferred that control reading of current passing across the nanoscale gap be measured prior to the addition of the sample. The current across the “sandwich” can then be compared to the control reading; changes in the measurement of electrical resistance, capacitance, impedance and/or conductance (as well as combinations thereof), compared to the blank control well, more reliably determines whether the sample is “positive” or “negative” for the analyte of interest. Although not shown in FIG. 1, it is generally preferred that the sample wells be thermostated (preferably at about 37° C.) to better calibrate and standardize the method (especially with regard timing and length of the incubation periods necessary for the various analytes of interest). Generally, incubations times for the capture antibodies and metal-labeled antibodies will be on the order of about 1 to about 5 minutes.

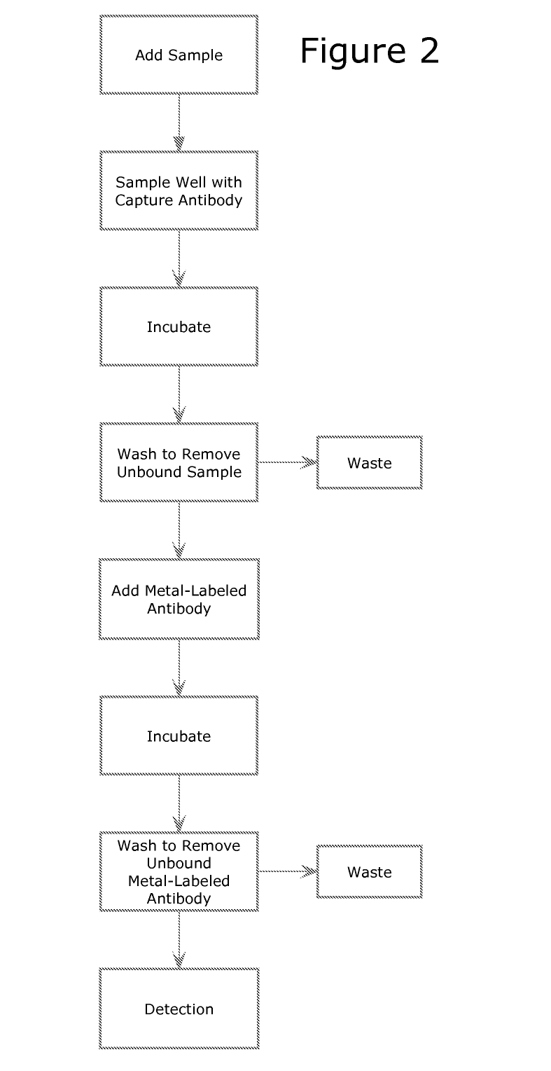

FIG. 2 provides a flowchart illustrating the method of the present invention. A sample is added to a sample well containing bound capture antibody which is specific to an epitope of the analyte of interest. The sample with the sample well is incubated for a time and at a temperature sufficient to allow the analyte, if present in the sample, to be bound to the capture antibody. The sample well is then treated with a wash solution to remove the sample and any excess analyte. A metal-labeled antibody, preferably a gold-labeled antibody, which is also specific to an epitope of the analyte is then added to the sample well. The materials within the sample well are then incubated for a time and temperature sufficient to allow the metal-labeled antibody to be bound to the analyte-capture antibody complex. The sample well is then treated with a second wash solution to remove any unbound metal-labeled antibody. If the analyte of interest was present in the sample, the sample well should now contain a metal-labeled antibody-analyte-capture antibody complex. If no analyte of interest was present in the sample, the sample well should be essentially free of the metal-labeled antibody. The change in the measurement of electrical resistance, capacitance, impedance and/or conductance (as well as combinations thereof), compared to the blank control well, determines the presence or absence of the analyte of interest.

As noted above, preferably the metal-labeled antibody and the capture antibody are specific to different epitopes on the analyte of interest. Indeed, the selection of specific epitopes used for the two antibodies can be used to increase the ability of the method to distinguish between analytes having at least some similar epitopes. Assuming that analyte 1 has major and epitopes A, B, C, and D and analyte 2 has major epitopes A, B, C, and E, it would be possible to distinguish between the two analytes using a capture antibody specific to epitope A for both analytes but metal-labeled antibodies specific to epitope D or epitope E, respectively.

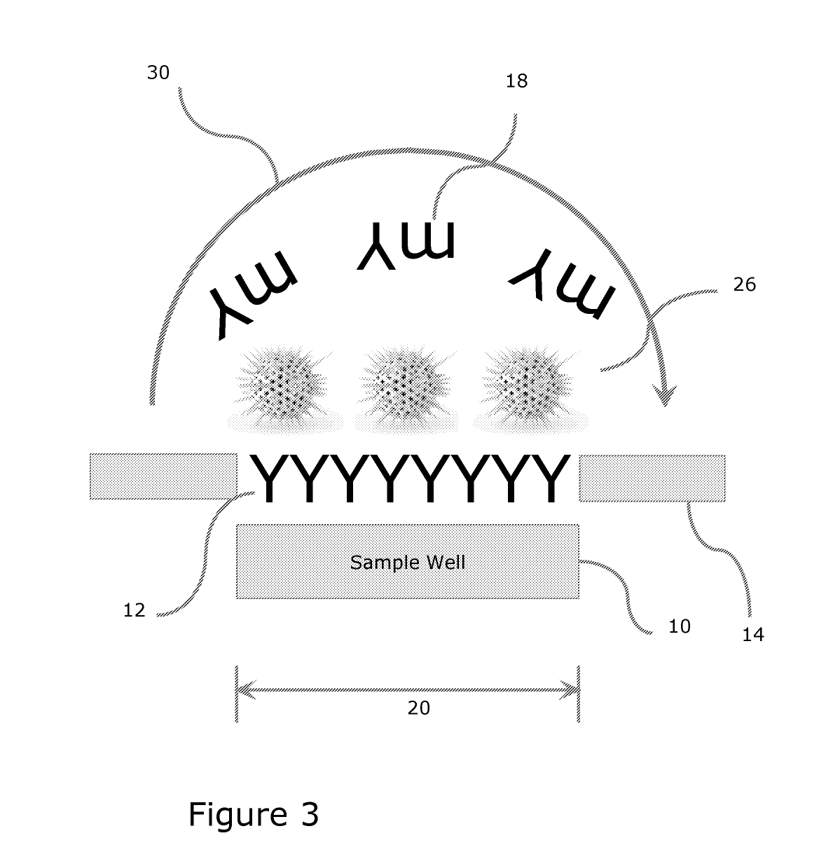

FIG. 3 provides a schematic view of a sample well 10 containing capture antibodies Y (12), analytes of interest 16, and metal-labeled antibodies mY (18). Electric current passed through electrodes 14 can electrically pass over the nanoscale gap 20 due to the conducting metal attached to the metal-labeled antibodies mY. Thus, if the analyte of interest is present in the sample, the electrical circuit can be completed over the nanoscale gap (shown as arrow 30) and the presence of the analyte of interest can be detected. Thus, a change in the measurement of electrical resistance, capacitance, impedance and/or conductance (as well as combinations thereof) of the sample well relative to a blank control well indicates the presence of the analyte of interest in the sample.

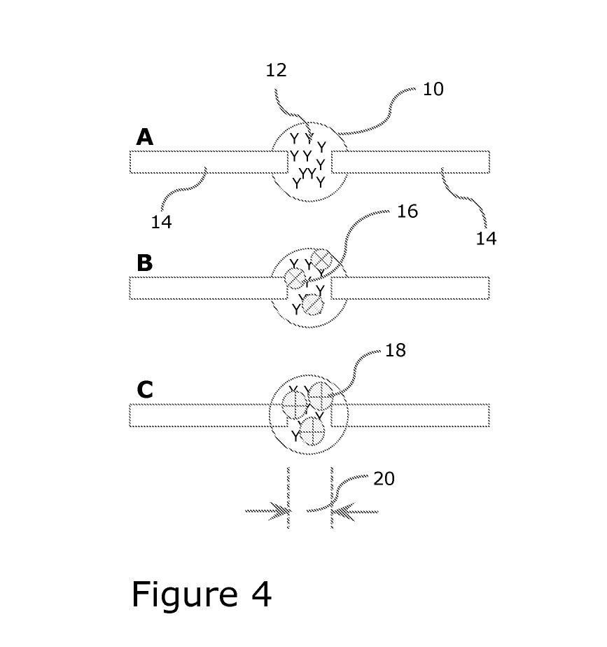

FIG. 4 provides a schematic view of the sample well 10 at various stages of the inventive method. FIG. 4A shows the sample well 10 containing capture antibody 12 prior to addition of the sample. Capture antibody 12 is specific to an epitope found on the analyte of interest. An electric current applied to electrode 14 will not be able to pass over the nanoscale gap 10. Attempts to pass an electric current over the sample well in FIG. 4A should result in little, if any, current passing across the nanoscale gap. Thus, measurements of electrical resistance, capacitance, impedance and/or conductance over the sample well in FIG. 4A can act a control as detailed herein. FIG. 4B shows the sample well 10 after exposure of the capture antibody 12 to the now-bound analyte of interest 16 in the sample. Analyte 16 (through its specific epitope) will bind to the capture antibody 12 embedded in the sample well 10. Finally, FIG. 4C shows the sample well 10 after exposure of the metal-labeled antibody 18 to the bound analyte-capture antibody complex. As noted above, the metal-labeled antibody 18 will bind through its specific epitope to the bound analyte-capture antibody complex. The metal bound to the antibody 18 will now allow the electric current to readily pass over the nanoscale gap 20 and allow detection of the analyte of interest.

The increase in sensitivity of the present method is due the nanoscale gap 20 across the sample well 10 and between the two circuits 14. Generally the nanoscale gap is in the range of about 10 to about 200 nanometers and preferable about 20 to about 75 nanometers. Within this nanoscale gap is imbedded certain commercially available or other monoclonal antibodies directed against a particular analyte of interest. The electrical resistance, capacitance, impedance and/or conductance can be measured over the sample well containing the bound capture antibody prior to the addition of the sample (or any other reagents) to provide a blank control well measurement. This blank control well measurement can be, and preferably is, used to compare the similar measurement taken after completion of the method of the present invention. After the control or blank measurement, the sample or specimen suspected of containing the analyte of interest is added. The analyte of interest, if present, will be captured by the antibodies 12 (preferably monoclonal antibodies) in the nanoscale gap. A series of timed incubations and washes initiated by the handheld detector actuators removes extraneous and nonspecifically bound proteins and contaminants leaving only the analyte of interest bound to the monoclonal antibodies in the nanoscale gap. A secondary metal-labeled antibody (again preferably monoclonal) directed to an epitope of the analyte of interest is then applied by actuating the platform well containing the secondary antibody. A series of timed incubations and washes removes non-specifically bound metal-labeled antibodies and the final reading is measured by the handheld device and compared to a control well. As noted above, the difference in the two wells determines whether the sample is “positive” or “negative” for the analyte of interest.

The secondary metal-labeled antibodies antibody contain elemental metal of high conductance such as, for example, gold, copper, silver, platinum, and the like. Preferably the metal is gold. As noted above, if analyte is present in the sample, a “sandwich” comprising the metal-labeled antibody, the analyte of interest, and the capture antibody will remain the sample well. The metal bound to the metal-labeled antibody will allow electrical current to pass over the nanoscale gap thereby indicating the presence of the analyte. Techniques for preparing such metal-labeled antibodies can be found in Ohashi et al. (“On-chip antibody immobilization for on-demand and rapid immunoassay on a microfluidic chip,” Biomicrofluidics 4, 032207 (2010); http://dx.doi.org/10.1063/1.3437592); which is hereby incorporated by reference in its entirety.

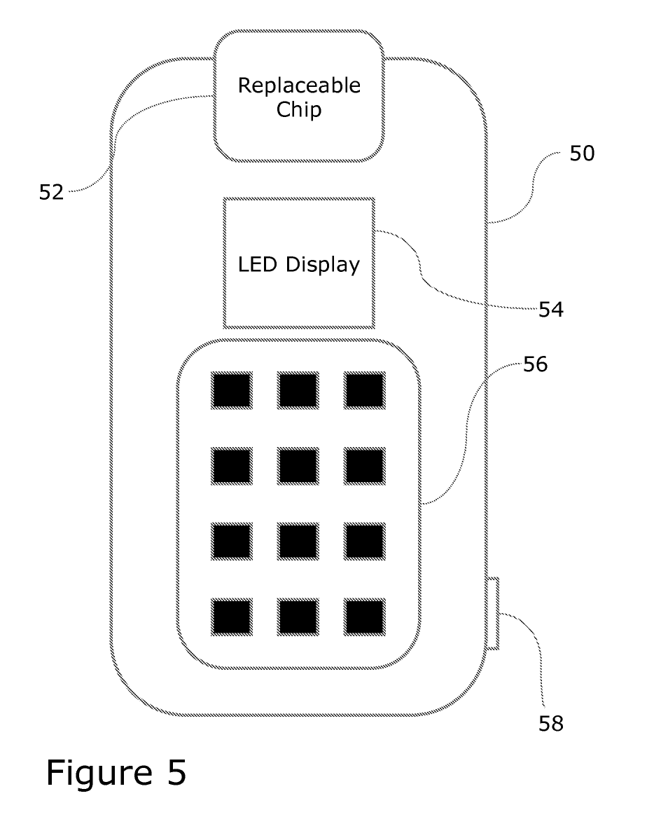

FIG. 5 illustrates one embodiment of the device of the present invention. The case 50, which is preferably hand-held and portable, contains an input means 56 to allow data regarding the sample and the like to be inputted, an LED display 54 by which the inputted data and ultimately the results can be viewed, and a replaceable chip or sample platform 52 which contains the means to carry out the method. The replaceable chip 52 contains the various sample wells with their bound capture antibodies. An output means 58 (e.g., blue tooth, USB outlet, or the like) is provided to allow inputted data and sample results to be downloaded to an appropriate device.

The platform chip or cartridge 52 may contain a multiplicity of wells for the same or different sample. Each well may contain the same antibodies directed against the same analyte of interest. Or a portion of the wells may contain different antibodies directed against different analytes of interest. For instance for an upper respiratory infection panel, there could be antibodies directed against different infectious agents that all cause the similar symptoms of upper respiratory infection but may require different course of treatment. For instance, in such an upper respiratory infection panel there could be antibodies directed against influenza type A and B viruses, parainfluenza types 1, 2, and 3 viruses, respiratory syncytial virus, rhinovirus, bacterial infectious agents, mycoplasma, or other fungal species. The detector would recognize the specific platform chip and read the appropriate results and indicate in a LED or electronic display a “positive” or “negative” result for each infectious agent tested. One advantage of multiplicity of wells on the same panel is to allow evaluation of a sample containing an unknown infectious agent or a sample potentially containing more than one infectious agent.

Preferable the hand-held portable detection device chips or cartridges contain the sample wells and all necessary reagents and wash solutions to perform the test in an automated manner. The sample, reagents, and wash solutions are preferably moved throughout the platform chips or cartridges as needed using microfluidic pumping techniques (e.g., microfluidic pumps, switches, channels, reservoirs, and the like).

For medical applications, the detection procedure of the present invention can be used with human and/or animal samples including, but not limited to, blood, serum, saliva, urine and the like. The detection procedure of the present invention can be also used for environmental testing of analytes such as toxins, pollutants, hazardous chemical compounds, and environmental organisms hazardous to human or animal health. Due to its increased sensitivity, the portable detection device of this invention can be used in the field or point of use applications to provide real-time results. Thus, the costs and delays associated with transporting specimens back to testing laboratories is avoided. Of course, if desired, the device of this invention can also be used in the field or point of use environment to determine samples showing positive results for particular analytes of interest which one may wish to transport back to the laboratory for more detailed analysis and study.

1. A device for detection of one or more analytes of interest in a biological or environmental sample, said device comprising
a device body and an interchangeable and replaceable platform chip or cartridge, wherein the device body is adapted to accept the interchangeable and replaceable platform chip;
wherein the device body comprises a battery, input means, and readout or output means;
wherein the platform chip or cartridge comprises
(1) a sample reservoir, a reservoir for a metal-labeled antibody, and at least one wash solution reservoir, wherein the metal-labeled antibody is specific to a first epitope of the one or more analytes of interest and the metal-labeled antibody contains an electrically conductive metal bound thereto;
(2) a plurality of sample wells containing an area of capture antibodies specific to a second epitope of the one or more analytes of interest, wherein the capture antibodies are bound to the sample wells;
(3) individual microelectrical circuits across each of the sample wells such that there is a nanoscale gap for each of the plurality of sample wells in the area of the capture antibodies, wherein the nanoscale gap is about 10 to about 200 nanometers; and
(4) at least one microfluidic pump adapted to individually move the sample, the metal-labeled antibody, and the one or more wash solution in a preselected order, through microfluidic channels into the sample wells at preselected times, hold within the sample wells for preselected periods, and remove from the sample wells at preselected times;
whereby the sample can be applied to the sample well to contact the capture antibodies and maintained therein for a sufficient time for analyte of interest, if present, to react with its corresponding capture antibody and be bound thereto to form an analyte-capture antibody complex, after which wash solution can be pumped through the sample well to remove unbound sample, after which metal-labeled antibody can be can be pumped into the sample well to contact any and maintained therein for a sufficient time for metal-labeled antibody to react with the analyte-capture antibody complex, if present, and be bound thereto form a metal-labeled antibody-analyte-capture antibody complex, after which wash solution can pumped through the sample well to remove unbound metal-labeled antibody, after which an electrical current can be passed across sample well such that if the analyte of interest was present, the metal-labeled antibody-analyte-capture antibody complex will allow the electrical current to pass over the nanoscale gap, thereby indicating the presence of the analyte of interest in the sample.

2. The device as defined in claim 1, wherein the capture antibody is a monoclonal antibody and the metal-labeled antibody is a monoclonal antibody, wherein the electrically conductive metal of the metal-labeled antibody is gold, and wherein the first and second epitopes on the analyte of interest are different.

3. A method for detecting of an analyte of interest in a biological or environmental sample, said method comprising
(1) applying the sample to a sample well containing bound capture antibodies, wherein the sample well has a microelectrical circuit attached to the sample well such that there is a nanoscale gap of about 10 to about 200 nanometers across the sample well in area of the capture antibodies, are wherein the bound capture antibodies are specific to a second epitope of the analyte of interest;
(2) maintaining the sample in contact with the bound capture antibody for a sufficient time for the analyte of interest, if present, to react with the capture antibody and be bound thereto to form an analyte-capture antibody complex;
(3) washing the sample well from step (2) to remove the unbound sample and leaving the analyte-capture antibody complex bound to the sample well;
(4) adding metal-labeled antibodies to the sample well in step (3), wherein the metal-labeled antibodies are specific to a first epitope of the analyte of interest and metal-labeled antibodies contain an electrically conductive metal bound thereto;
(5) maintaining the metal-labeled antibodies in contact with the analyte-capture antibody complex for a sufficient time for the analyte of interest, if present, to react with the analyte-capture antibody complex and be bound thereto to form a metal antibody-analyte-capture antibody complex;
(6) washing the sample well from step (5) to remove the unbound metal-labeled antibody and leaving the methal antibody-analyte-capture antibody complex bound to the sample well; and
(7) passing an electrical current across sample well and its microelectrical circuit such that if the analyte of interest was present, the metal-labeled antibody-analyte-capture antibody complex will allow the electrical current to pass over the nanoscale gap, thereby indicating the presence of the analyte of interest in the sample.

4. The method as defined in claim 3, wherein the capture antibody is a monoclonal antibody and the metal-labeled antibody is a monoclonal antibody, wherein the electrically conductive metal of the metal-labeled antibody is gold, and wherein the first and second epitopes on the analyte of interest are different.
